(19) United States
(12) Patent Application Publication
(10) Pub. No.: US 2025/0170540 A1
(43) Pub. Date: May 29, 2025

(54) DEVICE FOR SHAKING SAMPLES

(71) Applicant: Infors AG, Bottmingen (CH)

(72) Inventors: Alexander HAWRYLENKO, Bottmingen (CH); Johan DOPPLER, Lutter (FR)

(21) Appl. No.: 18/843,520

(22) PCT Filed: Mar. 3, 2023
(86) PCT No.: PCT/EP2023/055472

§ 371 Date: Sep. 3, 2024

(30) Foreign Application Priority Data
Mar. 4, 2022 (WO) PCT/EP2022/055638

(51) Int. Cl. B01F 31/22; B01F 31/20; B01F 35/42; B01F 101/23

(52) U.S. Cl. CPC B01F 31/22; B01F 31/265; B01F 35/42; B01F 2101/23

(57) ABSTRACT

A device for shaking samples includes a carrier (62) and a drive element (66) which is rotatably bearing-mounted on the carrier (62) about a drive axle and is drivable by a drive (65). Furthermore, the device includes a tray (61) arranged for loading the samples (69), a tray shaft (67) which is connected to the tray (61) and is bearing-mounted eccentrically on the drive element (66), and a counterweight (66a) which is attached to the drive element (66) and is adapted to compensate for an imbalance occurring during operation of the device with a defined load on the tray (61). A center of gravity of the counterweight (66a) is located opposite the tray shaft (67) in relation to the drive axle of the drive element (66). The center of gravity of the counterweight (66a) and a center of gravity of the tray (61) together with the tray shaft (67) and defined load are located in the same plane orthogonal to the drive axle. When the drive element (66) rotates about the drive axle, a first torque, which is exerted on the drive axle by the tray (61) together with the tray shaft (67) and defined load, is equal in magnitude and directed in the opposite direction to a second torque, which is exerted on the drive axle by the counterweight (66a). As a result, the counterweight (66a) is adapted in such a way that it compensates for both a static and a dynamic imbalance on the drive axle. Accordingly, a shaking frequency of the tray (61) due to the drive (65) reaches at least 1000 rpm, in particular at least 2000 rpm.

### CROSS-REFERENCE TO RELATED APPLICATIONS

This application is a National Stage application of International Patent Application No. PCT/EP2023/055472, filed on Mar. 3, 2023, which claims priority to International Patent Application No. PCT/EP2022/055638, filed on Mar. 4, 2022, each of which is hereby incorporated by reference in its entirety.

### TECHNICAL FIELD

The invention relates to a device for shaking samples, in particular a laboratory shaker, in particular for shaking and/or mixing samples containing liquid. The invention is advantageous for the cultivation of bacteria and cells in liquid culture media.

### BACKGROUND

Shakers are used to shake and/or mix liquids, e.g. cell cultures, biofuels or blood samples, in vessels in an orbital motion. The shaken unit often comprises a tray on which the vessels, e.g. Erlenmeyer flasks, test tubes or other ampoules, with the samples are located. A high shaking frequency is desirable for good mixing. At the same time, a high shaking frequency enables rapid oxygen transfer from the gas phase to the liquid phase, which facilitates good growth of the cell cultures.

However, particularly at high shaking frequencies, considerable imbalances occur on the rotating components during the shaking process, e.g. on a drive shaft and/or on a drive element on which the tray is bearing-mounted. Such an imbalance leads to vibrations of the shaker during the shaking process and thus to noise as well as to damaging forces on bearings with which the rotating components are bearing-mounted, which in turn leads to increased wear. In this respect, an imbalance on a rotating component limits the maximum achievable shaking frequency for continuous operation of the shaker in practice. As a result, the cultivation of cells in a conventional shaker only enables much slower growth and the production of significantly less biomass than cultivation in a bioreactor.

EP 3479894 A1 presents a shaker in which the tray with a tray shaft is bearing-mounted eccentrically in a drivable hollow shaft. A counterweight is provided on the hollow shaft to balance an imbalance caused by eccentric mass distribution. However, this counterweight (reference sign 17 in FIG. 2) has the disadvantage that it only partially compensates for an imbalance of the hollow shaft, as it only compensates for a static imbalance in particular. The task is therefore to provide a device for shaking samples which enables better mixing of the samples and/or sufficiently good mixing of larger samples. Alternatively, the task can be seen in providing a device for shaking samples which is suitable for continuous operation at a high shaking frequency above 1000 rpm, in particular above 1500 rpm, 2000 rpm or 2500 rpm, e.g. at 3 mm diameter of the orbital movement.

### SUMMARY

This problem is solved by a device for shaking samples, a so-called shaker, according to claim 1. By a sample is meant in particular a substance or a composition of substances which contains a liquid, e.g. a cell culture, a biofuel or a blood sample. The sample is usually contained in a vessel, e.g. a test tube or a microtiter plate, which in turn can be held by a vessel holder or stand.

The device comprises
a carrier: The carrier is especially designed to carry or support a tray.
a drive element that is bearing-mounted on the carrier so that it can rotate about a drive axle and can be driven by a drive, e.g. by a motor: The drive element can comprise a hollow shaft or a drive disk, for example.
a tray for loading the samples: In particular, the tray is a planar element on which the samples can be mounted. Advantageously, the tray comprises fastening elements for fastening at least one vessel with a sample or for fastening at least one vessel holder or stand.
a tray shaft, which is connected, in particular firmly, to the tray and is bearing-mounted eccentrically on the drive element: Advantageously, the tray shaft is attached centrally to the tray, in particular close to a center of gravity of the tray or of the tray with a defined load of samples. The term “close” refers in particular to an area around the center of gravity of up to +/−10% of the length and width of the tray. Due to the central attachment, the tray shaft (with a defined load) supports the tray close to the center of gravity.

The eccentricity (of the bearing) of the tray shaft on the drive element is the distance between an axis of rotation of the tray shaft and the drive axle of the drive element, which in particular run parallel to each other. The eccentricity determines the deflection of the tray (and thus the samples) during shaking. In particular, the eccentricity of the bearing of the tray shaft on the drive element can be between 0.5 and 3 mm, especially between 1 and 2 mm. This leads to a deflection of the tray which is adapted for shaking samples in smaller vessels, e.g. test tubes and microtiter plates.

The achievable shaking frequency of the tray as a result of the drive is at least 1000 rpm, in particular at least 1500 rpm, at least 2000 rpm or at least 2500 rpm. Shaking frequencies this high cannot be achieved with previous shakers, particularly due to imbalances. A shaking frequency in this range leads to good mixing of the samples, especially in smaller vessels, e.g. test tubes and microtiter plates, in particular with a volume of up to 1 ml. In addition, with a shaking frequency in this range, sufficient mixing of larger samples, in particular with a volume of more than 1 ml, e.g. 2 ml, can also be achieved, which is not possible with a lower shaking frequency. The shaking device according to the invention can therefore be used to cultivate larger quantities of cells, e.g. in higher vessels, which makes cultivation considerably more efficient.

Furthermore, the device comprises a counterweight attached to the drive element, which is adapted to compensate for an imbalance occurring during operation of the device with a defined load on the tray. The defined load of the tray with samples is characterized in particular by a mass, e.g. a number of samples times the average mass of the samples plus the mass of the vessels and any vessel holders, and a mass distribution on the tray, e.g. a distribution or center of gravity of the samples, vessels and any vessel holders on the tray. For good compensation of the imbalance, the mass and the mass distribution or the center of gravity of the load preferably deviate only insignificantly from the defined values, i.e. the defined load. With regard to the mass, this means in particular a deviation of at most +/−25%, with regard to the center of gravity, in particular a deviation of at most 3 cm.

An unbalance is defined in particular as a rotating body whose axis of rotation does not correspond to one of its main axes of inertia. A static unbalance occurs in particular when the axis of rotation does not run through the center of gravity of the body. Dynamic imbalance (also known as moment imbalance) occurs when the axis of rotation does not coincide with one of the main axes of inertia of the body, but is tilted in relation to the main axes of inertia at the center of gravity. In general, a body with any axis of rotation exhibits both static and dynamic unbalance. The unbalance can be clearly broken down into a static and a dynamic component. The static component leads to a rotating resultant force, while the dynamic component leads to a rotating resultant torque. Both the resulting force and the resulting torque increase quadratically with the speed.

According to the invention, the counterweight is adapted so that it compensates for both a static and a dynamic imbalance on the drive axle. Advantageously, the counterweight is the only counterweight on the drive element. In particular, there is no further counterweight on the side of the drive element opposite the tray. This is a difference to conventional unbalance compensation in shakers: A counterweight is conventionally only used to compensate for static unbalance, while another counterweight would be required to compensate for dynamic unbalance.

The device with the counterweight, which compensates for both a static and a dynamic imbalance on the drive axle, has several advantages: On the one hand, the resulting torque on the drive axle and in particular on its bearings can be avoided. This results in less noise emissions and less wear, especially at high shaking frequencies (in particular 1000 rpm and more), so that the service life of the device is increased. On the other hand, the device can be built compactly with the (single) counterweight, which is particularly advantageous for shakers with several trays on top of each other. In addition, high shaking frequencies, in particular of more than 1000, 1500, 2000 or 2500 rpm, can only be achieved with the described counterweight. This in turn leads to better mixing of the shaken samples and faster oxygen transfer from the gas phase to the liquid phase, which enables good growth of cell cultures in the samples.

### Counterweight

To compensate for the static and dynamic imbalance, the center of gravity of the counterweight is located opposite the tray shaft in relation to the drive axle of the drive element. As the weight of the tray and its load act eccentrically on the drive element via the tray shaft, the counterweight is attached to the drive element in such a way that the center of gravity of the counterweight is opposite the tray shaft in relation to the drive axle. In particular, the center of gravity of the counterweight and the tray shaft rotate around the drive axle when the drive element rotates. With a suitable choice of mass and center of gravity of the counterweight, the static imbalance can be compensated.

In addition, the center of gravity of the counterweight and a center of gravity of the tray together with the tray shaft and defined load are in the same plane, orthogonal to the drive axle. This prevents the occurrence of dynamic imbalance.

Furthermore, when the drive element rotates around the drive axle, a first torque, which is exerted on the drive axle by the tray together with the tray shaft and defined load, is equal in magnitude and directed in the opposite direction to a second torque, which is exerted on the drive axle by the counterweight. In this way, static and dynamic imbalance on the drive element can be avoided.

In one embodiment, the tray has a protrusion in the region of the tray shaft, within which the counterweight is at least partially located. In particular, the center of gravity of the counterweight is located inside the protrusion. This has the advantage that the counterweight as well as the other components, e.g. tray shaft and drive element, are protected from contamination, e.g. spilled or splashed samples, by the tray. This makes it easier to clean the device after contamination. At the same time, the protrusion and specific arrangement of the counterweight can ensure that the center of gravity of the counterweight and the center of gravity of the tray together with the tray shaft and defined load (as described above) are in the same plane.

Advantageously, the counterweight has a mass of between 0.1 and 1 kg, in particular between 0.5 and 0.9 kg. In addition, the counterweight can be made of metal, in particular cast iron or stainless steel. Due to its high density, such a counterweight can be compactly dimensioned and fitted in the protrusion to save space. In addition, such a counterweight only needs to be mounted at a distance of a few centimeters, in particular between 1 and 5 cm, e.g. between 2 and 3 cm, from the drive axle, for example to compensate for an imbalance with the above-mentioned advantageous eccentricities of between 0.5 and 3 mm, in particular between 1 and 2 mm, and a load on the tray of e.g. 15, 20 or 25 kg.

In one embodiment, the tray has a rectangular shape. This allows space-saving loading of the tray with typical containers and container holders. In particular, the tray can have a length of between 50 and 100 cm and/or a width of between 30 and 70 cm. Such a tray is suitable both for a table shaker, which is placed as a stand-alone device, e.g. on a laboratory bench, and for a multi-shaker with several trays on top of each other, which are enclosed in a housing, for example.

### Rotation Lock of the Tray

Advantageously, the tray is secured against rotation relative to the carrier in order to generate the desired orbital movement. Otherwise, the tray would also rotate around the drive axle when the drive element rotates. However, an orbital movement is desired for the shaking process, i.e. a translation of the tray together with the load on a circular path, which is given in particular by the eccentricity of the bearing of the tray shaft on the drive element. Such a translation can be achieved in various ways.

In one embodiment, the drive element has a first belt pulley for driving via a belt. In addition, the device comprises a second drive element and a second tray shaft, which is connected to the tray and bearing-mounted eccentrically on the second drive element. A counterweight for compensating the imbalance is also attached to the second drive element as described above. In particular, a center of gravity of the second counterweight is located in relation to the second drive axle of the second drive element opposite the second tray shaft. In addition, the center of gravity of the second counterweight and the center of gravity of the tray together with the second tray shaft and defined load are located in the same plane extending orthogonally to the second drive axle. Furthermore, when the second drive element rotates about the second drive axle, a third torque, which is exerted on the second drive axle by the tray together with the second tray shaft and defined load, is equal in magnitude and directed in the opposite direction to a fourth torque, which is exerted on the second drive axle by the second counterweight. Advantageously, the second drive element comprises a second belt pulley, which is connected to the first belt pulley via a toothed belt and is thus driven. Advantageously, the tray is bearing-mounted on the second drive element with the same eccentricity as on the drive element together with the first belt pulley, so that the tray is secured against rotation by the synchronous drive of the first and second belt pulleys.

In a particularly advantageous embodiment with first and second belt pulleys, first and second tray shafts and first and second drive elements, the tray comprises a first tray part and a second tray part. The first and second tray shafts are connected, in particular firmly, to the first and second tray parts and are bearing-mounted eccentrically on the first and second drive elements. To prevent rotation of the tray, the first and second tray parts are connected to each other by a linear guide. Such a linear guide is designed in particular to ensure that the first and second tray parts can move relative to each other along an axis of the linear guide, but cannot rotate relative to each other. In other words, the linear guide restricts all degrees of freedom of the tray parts in relation to each other with the exception of a degree of freedom of translation along the axis of the linear guide. The embodiment with two tray parts connected by a linear guide has the advantage that damaging forces on the bearing of the tray shafts, which are caused for example by thermal expansion of a one-piece tray, are prevented. This in turn enables higher shaking frequencies, a longer operating time and smoother running of the shaker.

Alternatively, rotation of the tray can also be prevented by the device additionally comprising flexible elements and/or articulated elements which are attached to the carrier or to a housing connected to the carrier and are designed to guide the tray. Advantageously, the flexible elements and/or articulated elements engage in an edge region of the tray.

### Housing

In one embodiment, the device additionally comprises an openable housing, wherein the tray, the tray shaft, the drive element and the carrier are located in an interior space within the housing. On the one hand, the housing can serve to seal off the tray and samples from an environment of the device, e.g. to prevent contamination of the environment by splashing liquid. On the other hand, the housing can be designed to air-condition the interior. For this purpose, the device can comprise a climate control element that is set up to control the temperature and/or humidity in the interior of the housing. For this purpose, and in particular to create ideal ambient conditions for the samples, the climate control element may comprise, for example, a heater, a cooler, a humidifier and/or a dehumidifier, which may be attached to the housing. To openably close the housing and thus maintain the ideal ambient conditions, the housing advantageously comprises a door, e.g. a hinged door, in particular on a front side of the housing.

### Several Trays

In one embodiment, the device comprises at least one further tray together with a further tray shaft, a further drive element, a further counterweight and a further carrier in the interior of the housing. In particular, the device can comprise at least five further trays, together with further tray shafts, further drive elements, further counterweights and further carriers in the interior of the housing. This increases the capacity of the device, i.e. in particular the number of samples that can be shaken simultaneously. Advantageously, the drive element and the at least one further drive element or the at least five further drive elements can be driven via a main drive shaft. The common drive via the main drive shaft results in a compact design that is easy to maintain.

To avoid an imbalance in the case of several trays, it is advantageous that an angular position of a bearing of the additional tray shaft on the additional drive element deviates from an angular position of the bearing of the tray shaft on the drive element. Otherwise, especially when the trays are heavily loaded, an imbalance may act on the main drive shaft and cause the entire device to vibrate.

This can be avoided in particular if the angular position of the bearings of the various tray shafts differ from each other by 360°/N, where N is the number of trays in the device. This compensates for any imbalance on the main drive shaft.

### Drive

In one embodiment, the drive comprises a motor, e.g. an electric motor, for driving the drive element and, if present, the at least one further drive element. In particular, the motor can be coupled to the main drive shaft via a gearbox.

Advantageously, the motor is mounted outside the housing if one is present. Mounting the motor outside the housing has the advantage that heat generated during operation of the motor is not introduced into the interior of the housing. For many applications or samples, it is desirable to control the temperature and/or humidity, e.g. by means of climate control as described above. In particular, the humidity is kept close to the dew point, especially at a relative humidity of between 80% and 100%. However, if the interior has to be cooled at the same time, e.g. due to the heat input from an engine in the interior, the humidity reaches 100% locally at the climate control or at the radiator and condenses out. Condensation is again undesirable because it can lead to an uncontrolled proliferation of foreign germs, which can be harmful to the samples. In particular, the interior of the housing should be thermally decoupled from the motor. This problem is solved by a motor mounted outside the housing.

In particular, the motor can be attached to the underside of the housing. This lowers the center of gravity of the device and thus increases the stability of the device, especially at high shaking frequencies. The main drive shaft is advantageously guided through an opening in the underside of the housing.

It should be noted that the various embodiments can also be combined where technically feasible, whereby synergistic effects and further advantages can be achieved.

### BRIEF DESCRIPTION OF THE DRAWINGS

Further embodiments, advantages and applications of the invention are apparent from the dependent claims and from the following description with reference to the figures. Showing:

FIG. 1a is a perspective view of a device for shaking samples according to one embodiment of the invention;

FIG. 1b is a top view of the device of FIG. 1a;

FIG. 2 is a schematic section through a device for shaking samples with an internal motor according to the state of the art;

FIG. 3 is a schematic sectional view of a device for shaking samples with an external motor according to one embodiment of the invention;

FIG. 4 is a schematic side view of an embodiment of the device according to the invention with several trays;

FIG. 5a is a schematic drawing of a device for shaking samples according to the state of the art, in which a static imbalance is compensated;

FIG. 5b is a schematic drawing of a device in which both a static and a dynamic imbalance are compensated;

FIG. 6a is a schematic vertical section through a device for shaking samples according to one embodiment;

FIG. 6b is a detailed view of region C of FIG. 6a;

FIG. 7 is a horizontal section or top view of a tray and a counterweight according to an embodiment of the invention;

FIGS. 8a, 8b and 8c are schematic drawings of a liquid sample in a vessel with increasing shaking frequencies;

FIG. 9 is a schematic section through a bearing with which the tray is bearing-mounted on the main drive shaft according to one embodiment;

FIG. 10a is a perspective view of a pivoting tray with a latching mechanism according to one embodiment;

FIG. 10b is an enlarged view of the latching mechanism in FIG. 10a;

FIG. 11a is an embodiment with a split tray in a perspective view;

FIG. 11b is an embodiment with a split tray in a schematic horizontal section; and

FIG. 11c is an embodiment with a split tray in a schematic vertical section.

### DETAILED DESCRIPTION

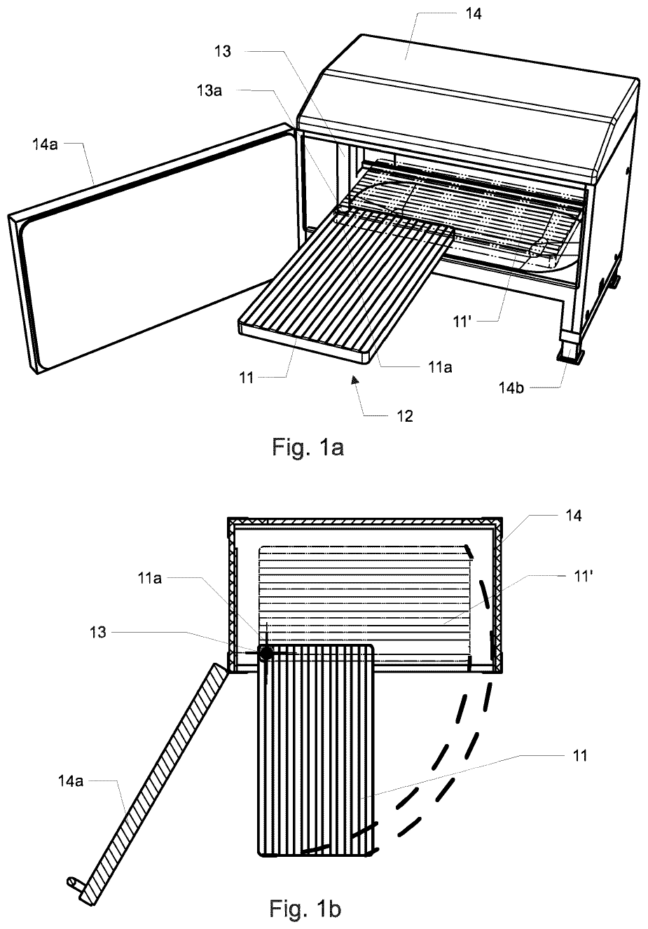

FIGS. 1a and 1b show a device for shaking samples, a so-called shaker, according to one embodiment. FIG. 1ais a perspective view of the device, while FIG. 1b shows a top view. The device comprises a tray 11, which is designed to be loaded with samples. The tray 11 is bearing-mounted on and supported by a carrier 12 (not visible in FIGS. 1a and 1b). The carrier 12 and with it the tray 11 can be pivoted about a main drive shaft 13. For this purpose, the carrier 12 is connected to the main drive shaft 13 via a bearing 13a, e.g. a ball bearing. In addition, the tray 11 has an opening 11a at its edge area, through which the main drive shaft 13 passes. The opening 11a is larger than a diameter of the main drive shaft 13, depending on the eccentricity of the bearing of the tray, in particular on the deflection of the shaking movement.

The tray 11 advantageously comprises an easy-to-clean surface, e.g. made of metal, at least on its upper side, i.e. the side facing the samples. This enables sterile operation of the device. Furthermore, the tray 11 can have a standard size of 850 mm×470 mm.

The tray 11 in the folded state 11′ and at least part of the main drive shaft 13 can be enclosed by a housing 14, which comprises a door 14a for opening and closing. The housing 14 generally fulfills several functions: Firstly, it forms a stationary frame which can be placed, for example via feet 14b, on a table, in a laboratory or generally on a base. The shaking movement of the tray takes place relative to this stationary frame. Secondly, the housing provides protection of the environment of the device, e.g. from splashing or spilling of samples or from vapors, which is particularly desirable for harmful samples or in a sterile laboratory. Thirdly, controlled conditions, e.g. in terms of temperature and/or humidity, can be set in an interior of the housing, as is advantageous for many samples. For this purpose, the device may comprise a climate control for the interior (as described above, not shown in FIGS. 1a and 1b).

In FIGS. 1a and 1b, the tray is shown in two positions: on the one hand (marked 11) swung out of the housing 14, and on the other hand (dashed, marked 11′) swung into the housing in an operational state. In the figure, the angle between the two positions is 90°. In general, however, an angle of at least 45° is already advantageous, as it improves the accessibility of the tray 11 and the interior of the housing 14.

Furthermore, it can be seen in FIGS. 1a and 1b that an attachment of the main drive shaft 13 in the edge region or even corner region of the tray, in particular less than 20% of a length and/or width of the tray away from an edge of the tray, is advantageous for the pivotability and accessibility of the tray. Alternatively, the same advantage can be achieved by the main drive shaft 13 extending outside the tray 11 close to its edge region, in particular less than 20% of the length or width of the tray away from the edge of the tray (not shown in FIGS. 1a and 1b). In this case, the opening 11a in the tray 11 is superfluous and the carrier 12 protrudes beyond the tray 11 in a horizontal direction. In general, the pivotability of the tray out of the housing improves the accessibility of the samples. In particular, a pivoting tray enables automation of the sample filling and removal process, as a robot arm, for example, can operate the device more easily under computer control.

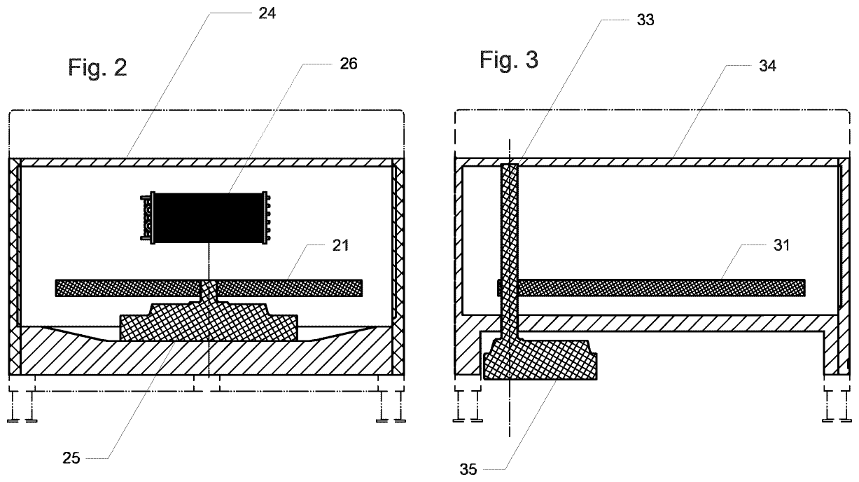

FIG. 2 shows a schematic sectional view of a shaker according to the state of the art. A tray 21 is driven by a motor 25, which is located inside a housing 24. The arrangement of the motor 25 in the interior of the housing 24 has the disadvantage that heat generated by the motor 25 directly heats up the interior and thus the samples located therein. For some samples, however, it is necessary to air-condition the interior, in particular to control the temperature and/or humidity, for example via an air conditioning control 26. As described above, this can lead to condensation of moisture in the interior, in particular on the air conditioning control 26 or the cooler, which in turn can damage the samples. An internal motor 25 contributes to this problem, as the heat generated by the motor 25 must be removed from the interior by the air conditioning control 26.

FIG. 3 illustrates a further aspect of the invention with a schematic section through a shaker. In contrast to FIG. 2 (prior art), the tray 31 can be driven here via a main drive shaft 33 by a motor 35, which is mounted outside the housing 34. The main drive shaft 33 runs orthogonally to the tray 31 and for the most part inside the housing 34, while a smaller part of the main drive shaft 33 runs outside the housing. The positioning of the motor 35 below the housing 34 is advantageous with regard to a low center of gravity of the device.

A motor mounted outside the housing, particularly as in FIG. 3, generally has the advantage that the heat generated by the motor is not introduced into the interior of the housing and therefore does not heat it up. This means that less cooling power is required to keep the interior at a constant temperature. As a result, less moisture condenses in the interior, e.g. locally on the climate control unit or the cooler, which could be harmful to the samples. This makes it easier to create controlled environmental conditions in the interior, in particular a constant temperature and high humidity, e.g. between 80% and 100% relative humidity without condensation.

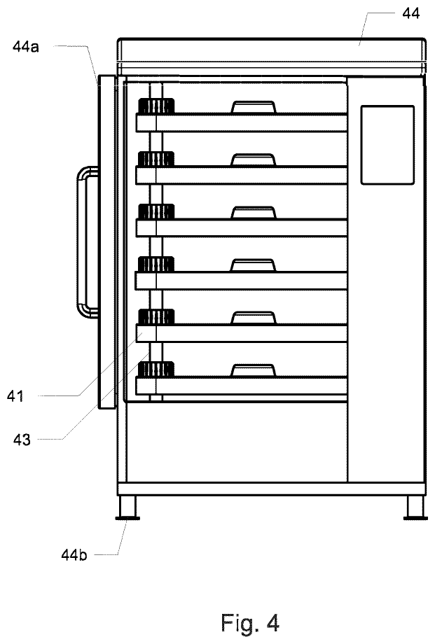

FIG. 4 illustrates a shaker with several, in particular six, trays 41 which are drivable in a housing 44 with door 44a by a single main drive shaft 43. Thus, all trays 41 are drivable by a motor (not shown in FIG. 4), which in turn may be mounted outside the possibly air-conditioned interior of the housing 44, as described above. Advantageously, the main drive shaft 43 again extends through the trays 41 in the edge region, in particular in the corner region, for optimum pivoting of the trays 41.

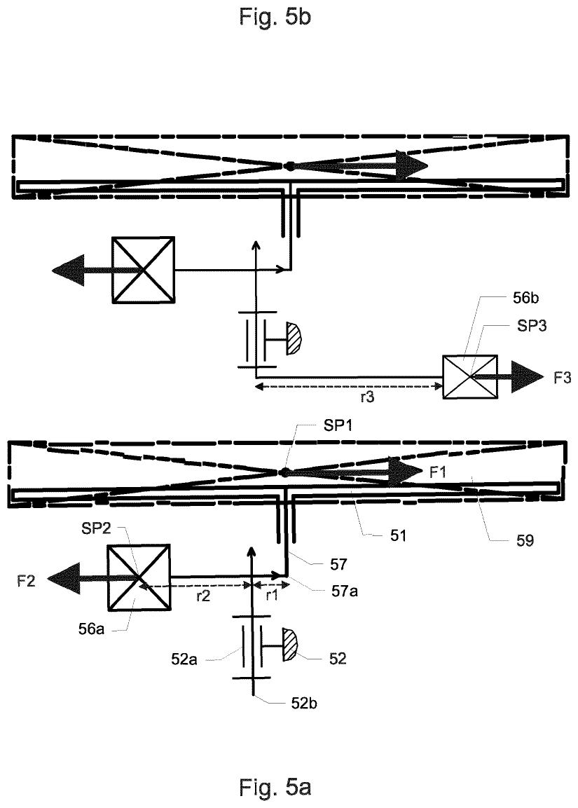

FIG. 5a shows by means of a schematic diagram how a static imbalance in a conventional shaker (as mentioned in the “Background” section above) can be compensated. A tray 51 with a load 59, comprising e.g. samples, vessels and vessel holders, is bearing-mounted eccentrically on a drive element, e.g. a hollow shaft, via a tray shaft 57. The bearing 57a of the tray shaft 57 on the drive element does not lie on the drive axle 52b, about which the drive element rotates due to its bearing 52a on a carrier 52. Rather, the bearing 57a is spaced from the drive axle 52b by the first radius r1 due to the eccentricity. The mass of tray 51, load 59 and tray shaft 57 therefore represents an unbalanced mass u1, which leads to the unbalance U1=u1*r1.

According to the prior art, a counterweight 56a is attached to the drive element to compensate for the static unbalance U1 in such a way that the center of gravity SP2 of the counterweight 56a is opposite the center of gravity SP1 of the unbalance mass u1 with respect to the drive axle 52b. In this case, the counterweight 56a is attached, for example, in the direction of the drive axle 52b between the center of gravity SP1 and the bearing 52a of the drive element. If the unbalance U2=u2*r2 of the counterweight 56a, with mass u2 of the counterweight and distance r2 of the center of gravity SP2 from the drive axle 52b, is the same size as the unbalance U1=U2, the static unbalance is just compensated. In other words, with the drive axle 52b horizontal, the drive element would not rotate from any initial position without the effect of an external force.

However, the arrangement with counterweight 56a according to FIG. 5a still has a dynamic unbalance, as the unbalances U1 and U2 are offset from each other by the center distance 1 in the direction of the drive axle 52b. When the drive element (including tray 51, load 59, tray shaft 57 and counterweight 56a) rotates, forces F1=U1*ω² and F2=U2*ω² act, see the thick arrows in FIG. 5a, where ω is the angular frequency. With U=U1=U2 and opposite directions of the forces F1 and F2, F=F1=−F2, these forces cause a torque M=F1*1/2−F2*1/2=1*F=1*U*ω² perpendicular to the drive axle 52b, which rotates with the drive axle. This results in a deviation torque D=u*1*r=U*1 when the static unbalance is ideally balanced.

FIG. 5b shows a schematic sketch of how the dynamic imbalance can also be compensated by an additional counterweight 56b. For this purpose, the additional counterweight 56b is mounted in the direction of the drive axle 52b on the other side of the bearing 52a as the tray 51 together with the load 59 and tray shaft 57 as well as the counterweight 56a. Furthermore, the additional counterweight 56b with mass u3, center of gravity SP3 and center distance r3 of the center of gravity SP3 in relation to the drive axle 52b is mounted on the same side as the center of gravity SP1, i.e. on the opposite side to the center of gravity SP2. As a result, an additional force F3 acts during rotation about the drive axle in the same way as above.

In this way, if u3 and r3 are selected appropriately, the dynamic imbalance on the drive axle 52b can be compensated for in addition to the static imbalance. This prevents damaging forces and wear of the bearing 52a. A disadvantage of the arrangement according to FIG. 5b, however, is the need to provide two counterweights 56a and 56b, which prevents a space-saving design.

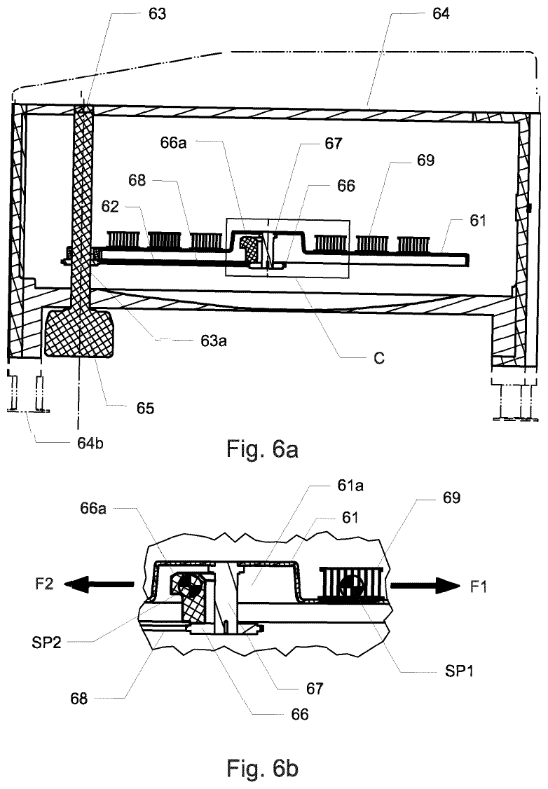

One aspect of the present invention therefore relates to a space-saving arrangement of a counterweight that compensates for both static and dynamic imbalance. Such an arrangement is described below with reference to FIG. 6b.

FIGS. 6a and 6b focus on the mechanical aspect of how a tray 61 is pivotably attached to the main drive shaft 63 via a carrier 62 with a bearing 63a (FIG. 6a), as well as details of the drive of the tray 61 via a drive element 66 with counterweight 66a (FIG. 6b). In principle, the mechanisms described can also be applied to several trays, e.g. to the shaker according to FIG. 4.

The tray 61 is set up to be loaded with one or more samples 69, for example in microtiter plates, which are to be shaken. For this purpose, the tray 61 preferably has fastening elements, e.g. for a vessel stand, in order to hold the samples 69 or vessels, in particular microtiter plates, stationary relative to the tray 61 during the shaking process.

The tray 61 is rotatably bearing-mounted on the drive element 66 via a fixed tray shaft 67. The drive element 66 is in turn rotatably mounted on the carrier 62, which is pivotably bearing-mounted on the main drive shaft 63 via the bearing 63a. The bearing of the tray shaft 67 in or on the drive element 66 is eccentric, so the axis of rotation of the tray shaft 67 does not coincide with the axis of rotation of the drive element 66. This eccentricity of the tray shaft 67 results in a circular movement when the drive element 66 rotates, on which the tray 61 rotates, and thus the desired shaking of the tray 61 together with the samples 69.

Optionally, the tray 61 is enclosed by a housing 64 when pivoted in, which serves as splash protection and/or for air conditioning the samples as described above. The main drive shaft 63 extends, in particular vertically, i.e. in the direction of gravity, through the housing 64 and is freely rotatably bearing-mounted on it. Furthermore, a motor 65 for driving the main drive shaft 63 is attached, preferably externally, to the housing 64.

In addition, the housing 64 (as already described with reference to FIG. 1a) may comprise feet 64b adapted to support the weight of the device. Generally, the feet may also be adapted for attachment to a support, such as a laboratory bench.

FIG. 6b is an enlarged view of section C in FIG. 6a. The tray 61, which can be loaded with samples 69, for example in a microtiter plate, is rotatably bearing-mounted on the drive element 66 via the tray shaft 67. The drive element 66 preferably comprises a belt pulley, which is driven by the main drive shaft via a belt 68. For this purpose, a second belt pulley is attached to the main drive shaft and the belt 68 is tensioned over the two belt pulleys. Advantageously, the tray 61 is also eccentrically bearing-mounted on the second belt pulley in the same way as on the first belt pulley on the drive element 66. This provides an anti-rotation lock for the tray 71, since its freedom of movement is thus restricted to a circular translation. In addition, as described above, the anti-rotation tray can comprise two tray parts which are connected to each other, for example by a linear guide.

It is generally advantageous (as described above) to compensate for imbalances that occur on rotating components of the device. In addition to the main drive shaft, cf. the section “Multiple trays” above, this applies above all to the drive element 66. Particularly at high shaking frequencies and therefore speeds, e.g. of over 1000 rpm, over 1500 rpm or even over 2000 rpm, as the device can reach, an imbalance otherwise leads to vibrations, to increased wear of the bearings and mountings and to excessive noise generation.

FIG. 6b shows an arrangement of a counterweight 66a on the drive element 66, which particularly simply and effectively compensates for an imbalance caused by the eccentric mounting of the tray 61 (with samples 69 and tray shaft 67) on the drive element 66. The center of gravity SP2 of the counterweight 66a is located in the same plane orthogonal to the axis of rotation of the drive element 66 as the center of gravity SP1 of the tray 61 together with the intended load of samples 69 and the tray shaft 67.

In general, the following applies: Since the two centers of gravity SP1 and SP2 lie in the same plane orthogonal to the axis of rotation of the drive element 66, the center distance 1=0 as defined above (for FIG. 5a). The two forces F1 and F2 that occur during rotation around the axis of rotation therefore act on the axis of rotation at the same point. This means that there is no deviation moment D=U*1=0 and the occurrence of a dynamic unbalance is prevented by design. If the condition for compensating a static unbalance (as described above for FIG. 5a) is also fulfilled, namely U1=U2, this is also avoided.

For the correction of the unbalance in practice, the above condition is considered to be fulfilled in particular if the unbalances U1 and U2 of tray 61 together with load 69 and tray shaft 67 on the one hand and of the counterweight 66a on the other hand deviate from each other by at most 25%. With respect to the center distance 1, i.e. the distance between the centers of gravity SP1 and SP2 in the direction of the axis of rotation, the above condition is considered to be fulfilled in particular if the center distance 1 is at most 1 cm. These tolerances allow the unbalance on the drive element 66 to be sufficiently compensated for in practice even in the event of minor deviations, for example with regard to mass or mass distribution, from the defined load.

According to FIG. 6b, this can be solved in such a way that the tray 61 comprises an upward protrusion 61a, under which at least a part of the counterweight 66a is located. The torques exerted by SP1 and SP2 when the drive element 66 rotates about the axis of rotation should generally just cancel each other out. With the arrangement shown, both a static and a dynamic imbalance can be compensated. This makes it possible to achieve high shaking frequencies of over 1000 rpm, in particular over 1500 rpm or over 2000 rpm, with a space-saving design. At the same time, this enables a long service life and continuous operation of the shaker by avoiding increased wear.

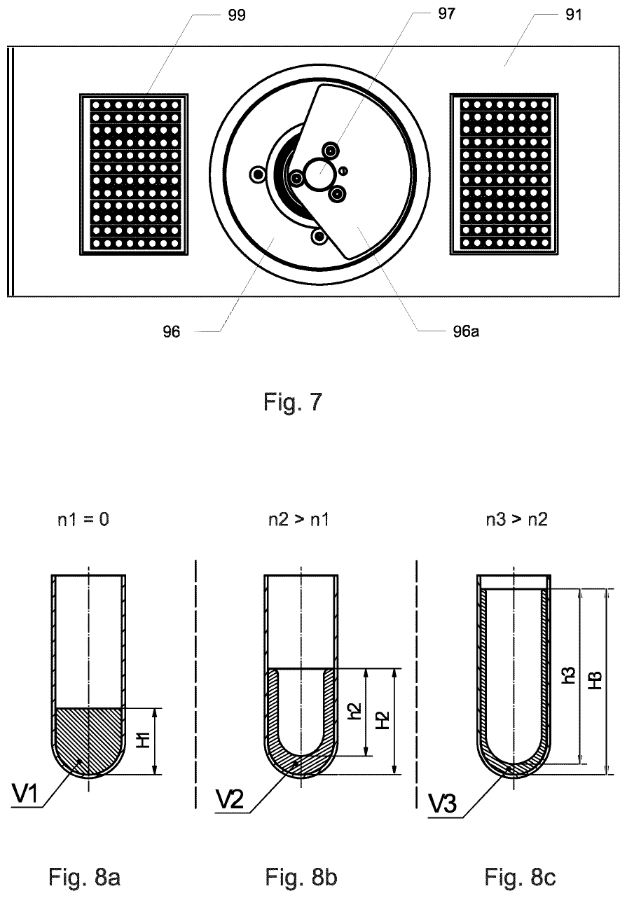

FIG. 7 shows a top view of or a horizontal section through a tray 91 with counterweight 96a. Two microtiter plates with a plurality of samples 99 are mounted on the tray 91, for example by means of vessel holders. As described above in connection with FIG. 6b, the counterweight 96a is attached to the drive element 96, so that an imbalance caused by the eccentric mounting of the tray 91 (with samples 99) on the drive element 96 is compensated particularly simply and effectively. As shown, the counterweight 96a can be attached to the drive element 96, for example with screws. Again, the center of gravity of the counterweight 96a is located in the same plane orthogonal to the axis of rotation of the drive element 96 as the center of gravity of the tray 91 together with the intended load of samples 99.

Advantageously, the counterweight 96a comprises an opening through which the tray shaft 97 extends. In addition, the counterweight 96a can advantageously be shaped similar to a sector of a circle when viewed from above. Both embodiments enable the largest possible volume and thus the largest possible mass of the counterweight 96a, whereby the counterweight 96a can nevertheless rotate with the drive element 96 in the protrusion of the tray 91. This maximizes the space available for the samples 99 on the tray 91.

FIGS. 8a, 8b and 8c illustrate the effect of different shaking frequencies n1, n2 and n3 on a liquid sample in a vessel, e.g. a test tube or a microtiter plate. The sample volume Vis the same in each of the illustrations: V1=V2=V3.

In FIG. 8a, the sample is at rest, i.e. shaking frequency n1=0. The sample liquid has an approximately flat and horizontal surface. The sample liquid fills the vessel to a height H1. This height can be taken as a measure of the diffusion distance dl that oxygen must travel from the surrounding gas into the sample: d1=H1.

In FIG. 8b, the sample is shaken at a shaking frequency n2>0, e.g. at n2=1000 rpm. The liquid is pressed upwards at the edge of the vessel and a meniscus, i.e. a concave surface of the sample liquid, is formed. While the surface area increases compared to the situation in FIG. 8a, the diffusion distance d2=H2−h2 decreases: d2<d1. Both result in oxygen entering the sample more quickly.

In FIG. 8c, the sample is shaken much faster, n3>n2, e.g. with n3=2000 rpm. The sample liquid is “pulled” far up the edge of the vessel. The surface area increases further due to the increasing meniscus and the diffusion distance d3=H3−h3 decreases further: d3<d2. Oxygen transport into the sample is therefore further improved.

This demonstrates one of the major advantages of the device described, which can achieve shaking frequencies of over 1000 rpm, in particular over 1500 rpm or over 2000 rpm: The oxygen transport from the gas phase into the liquid phase, i.e. into the sample, is greatly increased. In particular, cells can be cultivated with a similar rapid growth rate and produce a similar amount of biomass as when cultivated in a bioreactor. Such a device can therefore be used to carry out initial tests during cell cultivation under conditions similar to those later used in the mature process, particularly in the bioreactor.

In addition, FIGS. 8a, 8b and 8c show that higher vessels can be used at higher shaking frequencies and a correspondingly larger sample volume can be sufficiently mixed. This means that the sample volume can be at least doubled, e.g. from 1 ml to 2 ml per individual sample in a deep well plate, and the samples can still be sufficiently mixed and supplied with oxygen. This makes cell cultivation considerably less time-consuming and more productive.

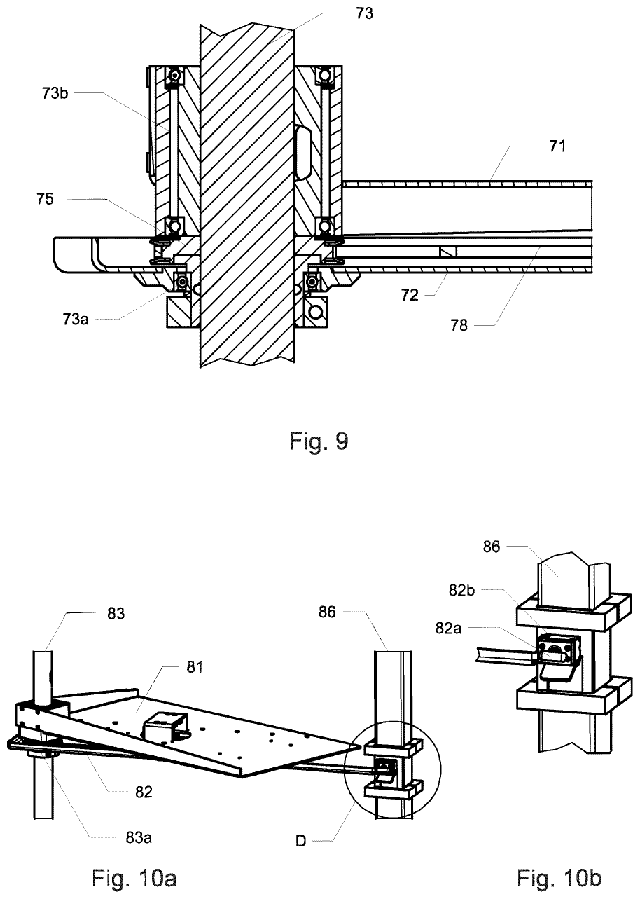

FIG. 9 shows how a tray 71 can be bearing-mounted on a main drive shaft 73 via a carrier 72. Such a mounting is compatible, for example, with the embodiments of FIGS. 1a/b, 3, 4 and 6a/b. As in FIGS. 6a/b, the tray 71 is bearing-mounted eccentrically on a drive element (not shown) with a first belt pulley. The drive element or the first belt pulley is rotatably bearing-mounted on the support 72 and is arranged to be driven via the belt 78. The belt 78 also runs over the second belt pulley 75, which is attached to the main drive shaft 73 and is accordingly driven by the drive or motor via the main drive shaft 73.

The carrier 72, which is designed to support the weight of the tray 71 together with the load of samples, is bearing-mounted on the main drive shaft 73 via a bearing 73a, e.g. a ball bearing. In this way, the carrier 72 can remain stationary, for example by being locked via a latch as in FIGS. 10a/b, while the main drive shaft 73 rotates. Preferably, the bearing 73a is located below the second belt pulley 75.

As an option, the carrier 72 together with the tray 71 can alternatively or additionally be bearing-mounted above the second belt pulley 75 via an additional bearing 73b on the main drive shaft 73. In general, care must be taken to ensure that the tray has sufficient clearance for its circular translation, which is caused by the eccentric bearing. In particular, an opening in the tray 71 through which the main drive shaft 73 passes in a preferred embodiment must be larger than the diameter of the main drive shaft 73 or than the second bearing 73b, if present, by at least the eccentricity of the bearing.

With a bearing 73a or 73b, several carriers can also be pivotably mounted one above the other on a main drive shaft 73 and driven simultaneously.

FIGS. 10a and 10b illustrate a possibility of locking a tray 81, which (as described, for example, in connection with FIGS. 6a/b and 9) is attached to a carrier 82 via a drive element (not visible), for the shaking process in the housing or on a support structure 86 in the housing, so that it is temporarily not pivotable about the main drive axle. The support structure 86 can be part of the housing or a separate component that is attached to the housing. For the pivoting of the tray 81, the support 82 is in turn pivotably bearing-mounted on the main drive shaft 83 via a bearing 83a, see e.g. FIG. 9.

FIG. 10b shows the section D of FIG. 10a enlarged. This comprises a first locking element 82a as a latch at or near its end remote from the main drive shaft. As a counterpart to the first locking element 82a, a second locking element 82b is attached to the support structure 86. The first and second locking elements 82a and 82b are in particular designed to establish a detachable connection upon contact. As a result, the carrier 82 can be connected to the support structure 86 for the shaking process and, in particular, pivoting of the carrier 82 about the main drive shaft 83 can be prevented. In addition, part of the weight of the carrier 82 and tray 81 together with the samples can be borne by the support structure 86, which reduces the load on the bearing 83a on the main drive shaft.

In general, the latching means comprises, for example, mechanical or magnetic components for releasably connecting the carrier 82 to the support structure 86. For example, the latching elements 82a and 82b may comprise magnets which are adapted to latch the carrier 82 to the support structure 86 by their mutual attraction. Alternatively, the locking elements 82a and 82b can be designed as a snap lock or as a hinged lock, in which a detachable connection is produced mechanically.

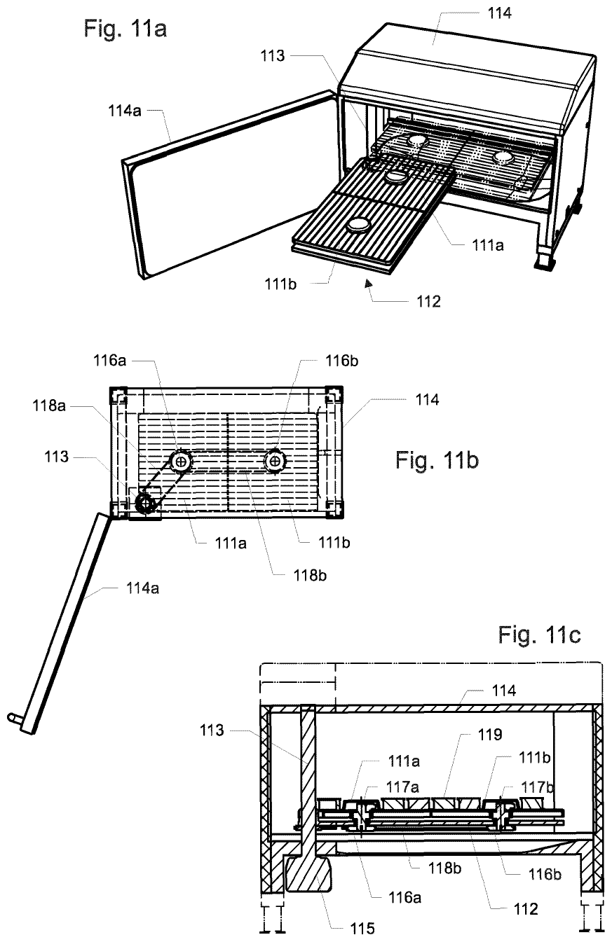

FIGS. 11a, 11b and 11c illustrate an embodiment of the shaker with a divided tray, whereby a particularly simple and reliable anti-rotation device for the tray can be achieved. FIG. 11a is a perspective view analogous to FIG. 1a; FIG. 11b is a schematic section through the shaker in the plane of the drive elements analogous to FIG. 1b; FIG. 11c is a schematic vertical section analogous to FIG. 6a. The features described for the previous embodiments are analogously applicable here.

The shaker comprises a housing 114 with a door 114a, which is arranged to open and close a front side of the housing. In FIGS. 11a and 11b, the door 114a is shown in the open state. Furthermore, the shaker comprises a main drive shaft 113, which can be driven by a motor 115. A carrier 112 is rotatably bearing-mounted on the main drive shaft 113, which can be engaged on the housing, for example by means of a latching mechanism (as described above). In turn, a first drive element 116a and a second drive element 116b are rotatably bearing-mounted on the carrier 112. The first drive element 116a is coupled to the main drive axle 113 via a first belt 118a and is driven by the latter. The second drive element 116b is coupled to the first drive element 116a via a second belt 118b and is thus also driven. It is important that the two drive elements 116a and 116b run synchronously. For this reason, a toothed belt is advantageously used at least for the second belt 118b.

A first tray shaft 117a or a second tray shaft 117b is eccentrically bearing-mounted on or in the first drive element 116a or the second drive element 116b. A first tray part 111a or a second tray part 111b is in turn attached to the first tray shaft 117a or to the second tray shaft 117b, in particular in a non-rotatable manner. Samples 119 can be placed on the tray parts 111a and 111b as described above, for example in test tubes or microtiter plates.

A particularly simple and reliable anti-rotation device for the two tray parts 111a and 111b can now be achieved by means of a flexible connection between the two tray parts (not shown in FIGS. 11a-c). Advantageously, this connection comprises a linear guide between the first tray part 111a and the second tray part 111b. The linear guide may, for example, be fixedly attached to one tray part while allowing the other tray part to slide along the guide. Such a flexible connection avoids damaging forces on the bearings of the tray shafts and drive elements, e.g. as a result of thermal expansion, in particular of the carrier 112.

Alternatively, the device can also comprise two drive elements 116a and 116b as shown in FIGS. 11a to 11c, which are driven synchronously without the tray being shown in two tray parts 111a and 111b. In this case, the tray is formed in one piece and (nevertheless as shown in FIGS. 11a to 11c) is bearing-mounted on the drive elements 116a and 116b via the first tray shaft 117a and 117b. In this case, the tray is also secured against rotation, so that in particular no additional guide elements, e.g. springs, are required between the tray and the housing 114. This in turn contributes to achieving the high speeds according to the invention.

While preferred embodiments of the invention are described in the present application, it should be clearly noted that the invention is not limited thereto and may be practiced in other ways within the scope of the following claims.

1. A device for shaking samples comprising
a carrier,
a drive element, which is rotatably bearing-mounted on the carrier about a drive axle and is drivable by a drive,
a tray arranged for loading the samples,
a tray shaft, which is connected to the tray and is bearing-mounted eccentrically on the drive element,
a counterweight attached to the drive element, which is adapted to compensate for an imbalance occurring during operation of the device with a defined load on the tray,
wherein a center of gravity of the counterweight is located opposite the tray shaft in relation to the drive axle of the drive element,
wherein the center of gravity of the counterweight and a center of gravity of the tray together with the tray shaft and defined load are located in the same plane extending orthogonally to the drive axle,
wherein, when the drive element rotates about the drive axle, a first torque, which is exerted on the drive axle by the tray together with the tray shaft and defined load, is equal in magnitude and directed in the opposite direction to a second torque, which is exerted on the drive axle by the counterweight,
wherein a shaking frequency of the tray due to the drive is at least 1000 rpm.

2. The device according to claim 1,
wherein the counterweight is adapted so that it compensates for both a static and a dynamic imbalance on the drive axle.

3. The device according to claim 1,
wherein the counterweight is the only counterweight on the drive element,
in particular wherein there is no further counterweight on the side of the drive element opposite the tray.

4. The device according to claim 1,
wherein the tray shaft is centrally attached to the tray.

5. The device according to claim 1,
wherein the tray has a protrusion in the region of the tray shaft,
wherein the counterweight is located at least partially within the protuberance.

6. The device according to claim 1,
wherein the counterweight has a mass of between 0.1 and 1 kg,
in particular wherein the counterweight is made of metal.

7. The device according to claim 1,
wherein an eccentricity of the bearing of the tray shaft on the drive element is between 0.5 and 3 mm, in particular between 1 and 2 mm.

8. The device according to claim 1,
wherein a shaking frequency of the tray as a result of the drive is at least 1500 rpm, in particular at least 2000 rpm or at least 2500 rpm.

9. The device according to claim 1,
wherein the tray has a rectangular shape,
in particular wherein the tray has a length of between 50 and 100 cm, and/or
in particular wherein the tray has a width of between 30 and 70 cm.

10. The device according to claim 1,
wherein the tray is secured against rotation relative to the carrier.

11. The device according to claim 10, further comprising
a second drive element, which is bearing-mounted on the carrier so as to be rotatable about a second drive axle and drivable by the drive, in particular wherein the second drive element is connected to the drive element via a toothed belt,
a second tray shaft, which is connected to the tray and is bearing-mounted eccentrically on the second drive element,
a second counterweight attached to the second drive element, which is adapted to compensate for an imbalance occurring during operation of the device with a defined load on the tray,
wherein a center of gravity of the second counterweight is located opposite the second tray shaft in relation to the second drive axle of the second drive element,
wherein the center of gravity of the second counterweight and the center of gravity of the tray together with the second tray shaft and defined load are located in the same plane extending orthogonally to the second drive axle,
wherein, when the second drive element rotates about the second drive axle, a third torque, which is exerted on the second drive axle by the tray together with the second tray shaft and defined load, of same magnitude and directed in opposite direction to a fourth torque, which is exerted on the second drive axle by the second counterweight.

12. The device according to claim 10,
wherein the tray comprises a first tray part and a second tray part,
wherein the first tray part and the second tray part are bearing-mounted eccentrically on a first drive element and a second drive element via a first tray shaft and a second tray shaft, respectively, which are connected to the first and second tray parts, respectively,
wherein the first drive element and the second drive element are rotatably bearing-mounted on the carrier and can be driven via the main drive shaft,
wherein the first and the second tray part are connected to each other by a flexible connection, in particular by a linear guide.

13. The device according to claim 1,
wherein the drive element comprises a belt pulley for driving via a belt.

14. The device according to claim 1, further comprising
an openable housing, in particular arranged for air-conditioning an interior of the housing,
wherein the tray, the tray shaft, the drive element and the carrier are located in the interior.

15. The device according to claim 1, further comprising
at least one further tray, in particular at least five further trays, together with a further tray shaft, further drive element, further counterweight and further carrier in the interior of the housing,
wherein the drive element and the at least one further drive element are drivable via a main drive shaft.

16. The device according to claim 14,
wherein the drive comprises a motor for driving the drive element and, if present, the at least one further drive element, in particular via the main drive shaft,
where the motor is mounted outside the housing,
in particular wherein the interior of the housing is thermally de-coupled from the motor.

17. The device according to claim 15,
wherein the drive comprises a motor for driving the drive element and, if present, the at least one further drive element, in particular via the main drive shaft,
where the motor is mounted outside the housing,
in particular wherein the interior of the housing is thermally de-coupled from the motor.
